(19) United States
(12) Patent Application Publication
(10) Pub. No.: US 2005/0181474 A1
(43) Pub. Date: Aug. 18, 2005

(54) Transport peptides and uses therefor

(76) Inventors: Frank J. Giordano, Madison, CT (US); William C. Sessa, Madison, CT (US)

Correspondence Address: FISH & NEAVE IP GROUP;ROPES & GRAY LLP, ONE INTERNATIONAL PLACE, BOSTON, MA, 02110-2624, US

(21) Appl. No.: 10/502,875

(22) PCT Filed: Jan. 30, 2003
(86) PCT No.: PCT/US03/02715

Related U.S. Application Data
(60) Provisional application No. 60/352,745, filed on Jan. 30, 2002.

(51) Int. Cl. C12P 21/06; C07H 21/04; C07K 7/06

(52) U.S. Cl. 435/69.1; 435/320.1; 435/325; 530/350; 530/329; 536/23.5

(57) ABSTRACT

The invention describes isolated transport peptides, which cross the cell membrane of a cell and/or home to a target cell. The invention also describes a transport complex in which a transport peptide is linked to a cargo moiety to be delivered into/to a cell. Methods are disclosed describing delivery of a transport complex into and/or to a cell. Vectors and host cells comprising transport peptides and transport complexes are also described, as well as pharmaceutical compositions including transport complexes of the present invention.

### RELATED APPLICATION

This application claims the benefit of the filing date of U.S. Provisional Application No. 60/352,745, entitled “Homing and Permeability Peptides to Facilitate Gene Delivery and Protein Transduction”, by Frank J. Giordano (filed Jan. 30, 2002). The entire teachings of the referenced Provisional Application are incorporated herein by reference.

### FUNDING

Work described herein was funded, in whole or in part, by National Institutes of Health grant HL 63770-01. The United States government has certain rights in the invention.

### BACKGROUND OF THE INVENTION

Clinically meaningful gene therapy protocols have yet to be developed. One of the greatest hindrances to the development of such gene therapy protocols is the problem of delivery. For example, the corrective genes for cystic fibrosis and muscular dystrophy have been known for years, but there has yet to be a successful gene therapy approach defined for either disease. One major reason for this is that no defined technology or methodology has been disclosed that facilitates the delivery of these corrective genes to enough cells in the body to effectively treat the disease.

More effective methods for the delivery of therapeutic proteins for the treatment of disease is also necessary. Peptides have been developed for many therapeutic uses. Delivery of the peptides into a cell, however, has remained problematic since they cannot readily cross biological membranes to enter cells. Current methods of peptide delivery into a cell include permeabilization of the cell membrane or microinjection into the cell. Both of these methods have serious drawbacks, however. Permeabilization of cells can only be practically useful for ex vivo methods, and these methods cause damage to the cells. Microinjection requires highly skilled technicians, it physically damages the cells, and it has only limited applications as it cannot be used to treat for example, a mass of cells or an entire tissue, because one cannot feasibly inject large numbers of cells.

There is a need for a more effective means of delivery of nucleic acids and proteins into cells for a variety of purposes, such as for the treatment of disease.

### SUMMARY OF THE INVENTION

The present invention relates to isolated peptides that cross the cell membrane of a cell. The invention also relates to isolated peptides that home to a cell and to isolated peptides that home to a cell and cross the cell membrane of that cell. Such peptides are herein referred to collectively as “transport peptides”. The isolated nucleic acids that encode these peptides are also the subject of this invention.

Isolated peptides that home to a cell and/or cross the cell membrane of a cell that are additionally linked to a moiety, herein referred to as a “cargo moiety”, to be delivered to/into a cell are also the subject of this invention. The term “transport complex” is used to refer to this embodiment of the present invention. The cargo moiety can be, for example, a protein, a nucleic acid molecule, a diagnostic agent, a prophylactic agent, or a therapeutic agent.

Expression vectors and isolated host cells comprising nucleic acid encoding a peptide that homes to and/or crosses the cell membrane of a cell and expression vectors and isolated host cells comprising nucleic acid encoding a cargo moiety linked to a peptide that homes to and/or crosses the cell membrane of a cell are also the subject of this invention. The invention additionally relates to methods of producing transport peptides and transport complexes.

The invention also relates to methods of use of the transport peptides and transport complexes of the invention. The invention relates to both in vitro and in vivo methods of delivering a cargo moiety to a cell and methods of importing a cargo moiety across the cell membrane into a cell. The invention also relates to in vitro and in vivo methods of delivering a cargo moiety to a cell and importing the cargo moiety across the cell membrane into the cell. The invention further relates to pharmaceutical compositions comprising a peptide that homes to and/or crosses the cell membrane of a cell linked to a cargo moiety.

The present invention provides peptides which deliver cargo moieties to a target cell and/or across a target cell membrane and thus is useful for delivery of cargo moieties, such as therapeutic proteins and nucleic acid molecules.

### BRIEF DESCRIPTION OF THE DRAWINGS

FIG. 1 depicts the amino acid sequences (SEQ ID NOS: 1-58) of transport peptides of the present invention.

FIG. 2 is a schematic of the in vitro functional biopanning approach taken to identify the transport peptides of the present invention.

FIGS. 3A and 3B are pictures depicting internalization of transport peptides labeled with rhodamine into cells in culture.

FIG. 3A is a picture showing uptake of a transport peptide labeled with rhodamine into endothelial (HUVEC) cells.

FIG. 3B is a picture showing uptake of a transport peptide labeled with rhodamine into smooth muscle cells.

FIGS. 4A-C are pictures depicting uptake of transport peptides labeled with rhodamine into endothelial cells in vivo.

FIG. 4A is a picture depicting virtually no uptake of a random (non-selected) peptide labeled with rhodamine.

FIGS. 4B and 4C are pictures depicting efficient uptake of transport peptides into the parenchyma of the heart after a single pass infusion through the coronary circulation.

FIG. 5 is a bar graph that depicts a reduction in VEGF driven vascular permeability in vivo due to a transport peptide fused to a caveolin peptide.

FIG. 6 is a bar graph that depicts results of a caveolin permeability assay.

### DETAILED DESCRIPTION OF THE INVENTION

Described herein are isolated peptides that cross the cell membrane of a cell. Also described are isolated peptides that home to a target cell, such as a specific cell type, and isolated peptides that home to and cross the cell membrane of a target cell. These peptides are herein collectively referred to as “transport peptides”. Nucleic acid (e.g., DNA, RNA) that encodes these isolated transport peptides is also an embodiment of this invention.

Isolated peptides of the present invention include, for example, isolated peptides having an amino acid sequence selected from the group consisting of: (a) GRKKDRA (SEQ ID NO: 1); (b) RATNRAH (SEQ ID NO: 2); (c) QRGGNQK (SEQ ID NO: 3); (d) RNNRRGG (SEQ ID NO: 4); (e) RRGR (SEQ ID NO: 5); (f) SSLVRTA (SEQ ID NO: 6); (g) GRTSPAR (SEQ ID NO: 7); (h) GGQANRS (SEQ ID NO: 8); (i) PVRNSRT (SEQ ID NO: 9); (j) PLGARNE (SEQ ID NO: 10); (k) RSGNR (SEQ ID NO: 11); (l) VIGGRSR (SEQ ID NO: 12); (m) HHGTTAR (SEQ ID NO: 13); (n) SKAPASE (SEQ ID NO: 14); (O) TAARGST (SEQ ID NO: 15); (p) CGRTRGA (SEQ ID NO: 16); (q) TGRSVGT (SEQ ID NO: 17); (r) RAATKCG (SEQ ID NO: 18); (s) LSGGQRS (SEQ ID NO: 19); (t) ATGAE (SEQ ID NO: 20); (u) LSNAPAG (SEQ ID NO: 21); (v) SGGLSGR (SEQ ID NO: 22); (w) HKRGGSS (SEQ ID NO: 23); (x) QGPTGAR (SEQ ID NO: 24); (y) DRRQSRH (SEQ ID NO: 25); (z) DRATRNS (SEQ ID NO: 26); (aa) GPGHAQF (SEQ ID NO: 27); (bb) APLRQGT (SEQ ID NO: 28); (cc) HRATERI (SEQ ID NO: 29); (dd) TTTAEGT (SEQ ID NO: 30); (ee) SALPHLL (SEQ ID NO: 31); (ff) RRPLHAT (SEQ ID NO: 32); (gg) PAHGLPP (SEQ ID NO: 33); (hh) IRLAGSA (SEQ ID NO: 34); (ii) LAARRSG (SEQ ID NO: 35); (jj) RRPRLRA (SEQ ID NO: 36); (kk) GPPHRLL (SEQ ID NO: 37); (ll) PLGAPAR (SEQ ID NO: 38); (mm) IVGTGRR (SEQ ID NO: 39); (nn) GLLVLKL (SEQ ID NO: 40); (oo) HQLRRVG (SEQ ID NO: 41); (pp) MRGAGRQ (SEQ ID NO: 42); (qq) AERGRAG (SEQ ID NO: 43); (rr) RRAGRTD (SEQ ID NO: 44); (ss) TKSRAGR (SEQ ID NO: 45); (tt) LLAVPAA (SEQ ID NO: 46); (uu) TIRAPGR (SEQ ID NO: 47); (vv) GPRVAHG (SEQ ID NO: 48); (ww) GPDRAPR (SEQ ID NO: 49); (xx) GLSLPPR (SEQ ID NO: 50); (yy) GSRHPPL (SEQ ID NO: 51); (zz) GAAPSRG (SEQ ID NO: 52); (aaa) GPQTRRL (SEQ ID NO: 53); (bbb) TALRLAT (SEQ ID NO: 54); (ccc) TSTALNL (SEQ ID NO: 55); (ddd) TVPGLML (SEQ ID NO: 56); (eee) TPVLTLH (SEQ ID NO: 57); and (fff) RRGRRRGR (SEQ ID NO: 58). Functionally equivalent variants of these peptides are also embodiments of this invention. Such variants include peptides with amino acid substitutions that maintain the functional integrity of the original peptide. Examples of amino acid substitutions include those that result in changes to the peptide wherein similar charge, polarity, hydrophobicity or structure of the original amino acid is maintained. Peptide variants also include peptide mimetics. Peptide mimetics include chemically modified peptides and peptide-like molecules containing non-naturally occurring amino acids.

Such peptides cross cell membranes and are useful to transport moieties to be delivered to/into cells. The transport peptides of the present invention are quite diverse and internalize into cells by different pathways (e.g., general membrane permeability vs. endocytosis vs. transcytosis vs. related receptor or adhesion compound-mediated transport). Blast searches of these motifs against international databases in some cases has yielded no similarity with known peptides. In other cases, such as with the peptide sequence LLAVPAA, (SEQ ID No: 46) we have found significant homology with known proteins. The sequence KKLLAVPAA, (SEQ ID NO: 59) for instance, is found routinely in receptors of the fibroblast growth factor family, as well as related tyrosine kinase receptors. The sequence LLAVPAA (SEQ ID No: 46) is also found in caveolin 2, and the leukotriene receptor. It is also found in a natural occurring permease. In none of these cases has this sequence been identified within those proteins as having a particular function, yet it is highly conserved among species. All of these known proteins are membrane bound and associated with endocytosis or transcytosis. This type of data supports a functional role of the transport peptides of the present invention related to the basis on which they were selected. In one embodiment, these transport peptides of the present invention will be useful as ‘bait’ in studies directed at further defining natural pathways by which macromolecules traffic into and out of cells.

For example, the peptide LLAVPAA (SEQ ID NO: 46) has been shown to be capable of translocating phage into heart, skeletal muscle, skin, and appears capable of crossing the blood brain barrier and entry into the brain.

In addition, there appears to be at least some degree of homing associated. GSRHPPL (SEQ ID NO: 51), for instance, appears to significantly target skin after intravenous delivery in vivo.

The terms “peptide” and “protein” as used herein refer to compounds made up of D- or L-amino acids or a mixture of D- and L-amino acids joined by peptide bonds. Generally, peptides contain at least two amino acid residues and are less than about 50 amino acids in length. In particular embodiments, transport peptides are 2 to 10, 5 to 10, 5 to 15, 4 to 12, 7 to 12, 10 to 20, 10 to 15 or 20 to 30 amino acid residues in length. “Polypeptide” as used herein refers to a polymer of at least two amino acid residues and which contains one or more peptide bonds. Polypeptide encompasses peptides and proteins. Amino acids are represented herein by their single letter codes.

A transport peptide of the present invention can be obtained from sources in which it occurs in nature or produced using known techniques, such as chemical synthesis or genetic engineering methods (e.g., recombinant DNA or RNA technology).

Isolated peptides of the present invention are relatively free from unrelated peptides as well as contaminating polypeptides, lipids, nucleic acids and other cellular material that normally are associated with the peptide in a cell or that are associated with the peptide in a library.

A cargo moiety of the present invention includes, but is not limited to, small molecules and macromolecules, such as polypeptides, nucleic acids and polysaccharides. The cargo moiety can be a nucleic acid molecule, such as DNA or RNA; a nucleic acid analog, such as peptide nucleic acid (PNA); a peptide; a protein; an oligosaccharide; a lipid; a glycolipid; a lipoprotein; a virus, such as T-7 bacteriophage; a biologically active compound; a drug; a label; an imaging agent; a diagnostic agent; a therapeutic agent; and a prophylactic agent.

The cargo moiety can be an organic molecule or compound or an inorganic molecule or compound. An organic molecule can be a drug; a nucleic acid molecule (e.g., DNA or RNA); a peptide; a variant or modified peptide or a peptide mimetic; a protein or a fragment thereof; an oligosaccharide; a lipid; a glycolipid; or a lipoprotein.

An organic molecule or compound can be obtained from a source in which it occurs in nature (e.g., from cells in which it occurs) or can be produced using known methods, such as genetic engineering methods (e.g., recombinant DNA or RNA technology) or chemical synthetic methods. For example, an organic molecule can be an RNA molecule, polypeptide or a fragment thereof, which can be isolated from a cell, expressed from a recombinant nucleic acid molecule or synthesized chemically.

An organic molecule also can be a non-naturally occurring molecule. Such molecules have chemical groups or bonds that are not normally produced by biological processes. For example, a nucleic acid sequence containing non-naturally occurring nucleoside analogs or phosphorothioate bonds that link the nucleotides and protect against degradation by nucleases are examples of non-naturally occurring molecules. A ribonucleotide containing a 2-methyl group, instead of the normal hydroxyl group, bonded to the 2′-carbon atom of ribose residues, is an example of a non-naturally occurring RNA molecule that is resistant to enzymatic and chemical degradation. Other examples of non-naturally occurring organic molecules include RNA containing 2′-aminopyrimidines, such RNA being 1000 times more stable in human serum and urine as compared to naturally occurring RNA (see Lin et al., Nucl. Acids Res., 22:5229-5234 (1994); and Jellinek et al., Biochemistry, 34:11363-11372 (1995), each of which is incorporated herein by reference).

In one embodiment of the present invention, the cargo moiety is DNA or RNA or a nucleic acid analog. The DNA or RNA can be an oligonucleotide of any length. Such nucleic acid molecules can be linear, circular or supercoiled, and can be single stranded or double stranded DNA or RNA or can be a DNA/RNA hybrid. Nucleic acid analogs include charged and uncharged backbone analogs, such as phosphonates (e.g., methyl phosphonates), phosphoramidates (N3′ or N5′), thiophosphates, uncharged morpholino-based polymers, and peptide nucleic acids (PNAs). Such molecules can be used in a variety of therapeutic regimens, including enzyme replacement therapy, gene therapy, and anti-sense therapy, for example.

By way of example, peptide nucleic acids (PNAs) are analogs of DNA. The backbone of a PNA is formed by peptide bonds rather than phosphate esters, making it well-suited for anti-sense applications. Since the backbone is uncharged, PNA/DNA or PNA/RNA duplexes that form exhibit greater than normal thermal stability. PNAs have the additional advantage that they are not recognized by nucleases or proteases. In addition, PNAs can be synthesized on an automated peptides synthesizer using standard t-Boc chemistry. The PNA can be linked to a transport peptide of the present invention using known methods.

Isolated nucleic acids of the present invention are relatively free from unrelated nucleic acids as well as contaminating polypeptides, nucleic acids and other cellular material that normally are associated with the nucleic acid in a cell or that are associated with the nucleic acid in a library.

In one embodiment of the invention, the cargo moiety is a polypeptide. In a certain embodiment, the cargo moiety is caveolin or a fragment thereof. In another embodiment, the cargo moiety is a transcription factor or a nuclear localization peptide. In a further embodiment, tow cargo moieties—one a transcription factor and the other a nuclear localization peptide—are present in a transport complex.

In another embodiment of the invention, the cargo moiety is a label, such as a dye. In a certain embodiment, the cargo moiety is the fluorescent marker, rhodamine. In other embodiments, the cargo moiety may be a marker, such as green fluorescent protein, blue fluorescent protein, yellow fluorescent protein or biotin.

The cargo moiety can be combined with or attached to the transport peptide to form the transport peptide-cargo moiety which is a subject of the present invention. The term “transport complex” is used to refer to this embodiment of the invention. The transport peptide and the cargo moiety are joined (by any means which produce a link (between the components) in such a manner that they remain joined under the conditions in which the transport complex is used (e.g., under conditions in which a transport complex is administered to an individual).

In one embodiment, the link between the transport peptide and the cargo moiety. Alternatively, the link can be a noncovatent association, such as electrostatic interaction is covalent. For example, recombinant techniques can be used to covalently attach a transport peptide to a cargo moiety, such as by joining DNA or RNA coding for the transport peptide with DNA or RNA coding for the cargo moiety and expressing the encoded products in an appropriate host cell (a cell capable of expressing the transport complex). Alternatively, the two separate nucleotide sequences can be expressed in a cell or can be synthesized chemically and subsequently joined, using known techniques. Alternatively, the transport peptide-cargo moiety can be synthesized chemically as a single amino acid sequence and, thus, joining is not needed.

“A cargo moiety” is interpreted to mean one or more than one cargo moieties linked to the transport peptide. In instances wherein there are more than one cargo moieties linked to the transport peptide, the moieties may be the same or different. The cargo moiety or moieties may be linked to the transport peptide at either the N- or C-terminus of the transport peptide. In embodiments wherein there are at least two cargo moieties linked to the transport peptide, one cargo moiety may be at the N-terminus of the transport peptide and one cargo moiety may be at the C-terminus of the transport peptide. Alternatively, more than one cargo moiety may be linked to either the N- or C-terminus of the transport peptide.

The cargo moiety can be linked to a peptide of the present invention either directly or indirectly by means of a linker. Linkers include, for example, one or more amino acid residues. The linker moiety may be, for example, a short sequence of amino acid residues (e.g., 1 to 10, 1 to 5 or 1 to 4 amino acid residues) the linker can optionally include a cysteine residue through which the linker moiety binds to the transport peptide or cargo moiety of the transport complex. A linker may also be, for example, an intermediary bonding group such as a sulphydryl or carboxyl group or any larger group. Suitable linking moieties include bi- and multi-functional alkyl, aryl, aralkyl or peptidic moieties, alkyl, aryl or aralkyl aldehydes, acids, esters and anhydrides, sulphydryl or carboxyl groups, such as maleimido benzoic acid derivatives, maleimido proprionic acid derivatives and succinimido derivatives or may be derived from cyanuric bromide or chloride, carbonyldiimidazole, succinimidyl esters or sulphonic halides. The functional groups on the linker moiety used to form covalent bonds between linker and cargo moiety on the one hand, as well as linker and transport peptide on the other hand, may be two or more of e.g., amino, hydrazine, hydroxyl, thiol, maleimido, carbonyl, and carboxyl groups, etc. In use, the transport complex may dissociate by way of chemical or enzymatic cleavage between the cargo moiety and transport peptide. Within the embodiments wherein the linker includes amino acid residues, such cleavage may occur within the linker itself.

In one embodiment, wherein the cargo moiety is a polypeptide, the cargo moiety is linked to the transport peptide as a fusion protein by means of recombinant technology. A fusion protein is the co-linear, covalent linkage of two or more proteins via their polypeptide backbones, through genetic expression of a nucleic acid molecule encoding those proteins. The nucleic acid encoding the cargo moiety of the fusion protein is in-frame with the nucleic acid encoding the transport peptide. “In-frame” is interpreted to mean that the nucleic acid sequence encoding the cargo moiety will be in the correct reading frame as will the nucleic acid sequence encoding the transport peptide. Therefore, the correct amino acid sequences will be translated for both the transport peptide and cargo moiety of the fusion protein.

In another embodiment, the cargo moiety is conjugated to the transport peptide via chemical cross-linking. Numerous chemical cross-linking methods are known and potentially applicable for linking the transport peptides of this invention to a cargo moiety. Coupling of the cargo moiety and the transport peptide can be accomplished via a coupling or linking agent. There are several intermolecular cross-linking reagents which can be utilized (see, for example, Means, G E and Feeney, R E Chemical Modification of Proteins, Holden-Day, 1974, pp. 39-43). Among these reagents are, for example, J-succinimidyl 3-(2-pyridyldithio) propionate (SPDP) or N,N′-(1,3-phenylene) bismaleimide (both of which are highly specific for sulphydryl groups and form irreversible linkages); N,N′-ethylene-bis-(iodoacetamide) or other such reagent having 6 to 11 carbon methylene bridges (which are relatively specific for sulphydryl groups); and 1,5-difluoro-2,4-dinitrobenzene (which forms irreversible linkages with amino and tyrosine groups). Other cross-linking reagents useful for this purpose include: p,p′-difluoro-m, m′-dinitrodiphenylsulfone (which forms irreversible cross-linkages with amino and phenolic groups); dimethyl adipimidate (which is specific for amino groups); phenol-1,4-disulfonylchloride (which reacts principally with amino groups); hexamethylenediisocyanate or diisothiocyanate, or azophenyl-p-diisocyanate (which reacts principally with amino groups); glutaraldehyde (which reacts with several different side chains) and disdiazobenzidine (which reacts primarily with tyrosine and histidine).

Many cross-linking reagents may yield a transport complex that is essentially non-cleavable under cellular conditions. However, some cross-linking reagents contain a covalent bond, such as a disulfide, that is cleavable under cellular conditions. For example, dithiobis(succinimidylpropionate) (“DSP”), Traut's reagent and N-succinimidyl 3-(2-pyridyldithio) propionate (“SPDP”) are well-known cleavable cross-linkers. The use of a cleavable cross-linking reagent permits the transport peptide to separate from the cargo moiety after delivery into the target cell. Direct disulfide linkage may also be useful.

Some cross-linking reagents such as n-γ-maleimidobutyryloxy-succinimide ester (“GMBS”) and sulfo-GMBS, have reduced immunogenicity. In some embodiments of the present invention, such reduced immunogenicity may be advantageous.

Numerous cross-linking reagents, including the ones discussed above, are commercially available. Detailed instructions for their use are readily available from the commercial suppliers. A general reference on protein cross-linking preparation is: S. S. Wong, Chemistry of Protein Conjugation and Cross-Linking, CRC Press (1991).

In another embodiment of the present invention, wherein the transport complex is a fusion protein. Expression system vectors, which incorporate the necessary regulatory elements for protein expression, as well as restriction endonuclease sites that facilitate cloning of the desired sequences into the vector, are known to those of skill in the art. A number of these expression vectors are commercially available.

A recombinant DNA expression vector containing the elements previously described is introduced into an appropriate host cell (a cell capable of expressing the transport complex) where cellular mechanisms of the host cell direct the expression of the fusion protein encoded by the recombinant DNA expression vector. Alternately, cell-free systems known to those of skill in the art can be chosen for expression of the fusion protein.

The purified fusion protein produced by the expression vector host cell system can then be administered to the target cell, where the transport peptide mediates the import of the fusion protein through the cell membrane of the target cell into the interior of the cell. A target cell is a specific cell type such as, for example, a cardiac cell, a skin cell, such as an epithelial cell; a skeletal muscle cell or a brain cell (e.g., a neuron), but may be any cell, including human and nonhuman cells.

An expression vector host cell system can be chosen from among a number of such systems that are known to those of skill in the art. In one embodiment of the invention, the fusion protein can be expressed in isolated host cells, such as Escherichia coli. In alternate embodiments of the present invention, fusion proteins may be expressed in other bacterial expression systems, viral expression systems, eukaryotic expression systems, or cell-free expression systems. Cellular hosts used by those of skill in the art include, but are not limited to, isolated host cells such as, for example, Bacillus subtilis, yeast such as Saccharomyces cerevisiae, Saccharomyces carlsbergenesis, Saccharomyces pombe, and Pichia pastoris, as well as mammalian cells such as NIH3T3, HeLa, HEK293, HUVEC, rat aortic smooth muscle cells and adult human smooth muscle cells. The expression vector chosen by one of skill in the art will include transcriptional activation elements such as promoter elements and other regulatory elements appropriate for the host cell or cell-free system in which the fusion protein will be expressed. In mammalian expression systems, for example, suitable expression vectors can include DNA plasmids, DNA viruses, and RNA viruses. In bacterial expression systems, suitable vectors can include plasmid DNA and bacteriophage vectors.

Examples of specific expression vector systems include the pBAD/gIII vector (Invitrogen, Carlsbad, Calif.) system for protein expression in E. coli, which is regulated by the transcriptional regulator AraC.

An example of a vector for mammalian expression is the pcDNA3.1 V5-His-TOPO eukaryotic expression vector (Invitrogen). In this vector, the transport complex can be expressed at high levels under the control of a strong cytomegalovirus (CMV) promoter. A C-terminal polyhistidine (6×His) tag enables transport complex purification using nickel-chelating resin. Secreted protein produced by this vector can be detected using an anti-His (C-term) antibody.

A baculovirus expression system can also be used for production of a transport complex comprising the transport peptide and a cargo moiety wherein the cargo moiety is a polypeptide. A commonly used baculovirus is AcMNPV. Cloning of the transport complex DNA can be accomplished by using homologous recombination. The transport complex DNA sequence is cloned into a transfer vector containing a baculovirus promoter flanked by baculovirus DNA, particularly DNA from the polyhedrin gene. This DNA is transfected into insect cells, where homologous recombination occurs to insert the transport complex DNA into the genome of the parent virus. Recombinants are identified by altered plaque morphology.

Many transport complexes in which the cargo moiety is a peptide or protein may not be appropriately post-translationally modified in bacterial expression systems can be expressed with baculovirus vectors. Enzymes, signaling molecules, mediators of cell cycle control, transcription factors, antigenic peptides, full-length protein products of viral, bacterial, or other origin for use in vaccine therapy, protein products of human cells for use in cancer vaccine therapy, toxins, and proteins involved in intracellular signaling systems which may not be appropriately post-translationally modified in bacterial expression systems can be expressed with baculovirus vectors.

Proteins as described above can also be produced by the method of the present invention by mammalian viral expression systems. An ecdysone-inducible mammalian expression system (Invitrogen, Carlsbad, Calif.), described by No, et al. (1996) can also be used to express the transport complex wherein the transport complex is a fusion protein.

In another embodiment of the invention, yeast host cells, such as Pichia pastoris, can also be used for the production of a transport complex by the method of the present invention. Expression of heterologous proteins from plasmids transformed into Pichia has previously been described by Sreekrishna, et al. (U.S. Pat. No. 5,002,876, incorporated herein by reference). Vectors for expression in Pichia of a fusion protein comprising a transport peptide of the present invention and a cargo moiety wherein the cargo moiety is a peptide or protein are commercially available as part of a Pichia Expression Kit (Invitrogen, Carlsbad, Calif.).

Purification of heterologous protein produced in Pichia has been described by Craig, et al. (U.S. Pat. No. 5,004,688, incorporated herein by reference), and techniques for protein purification from yeast expression systems are well known to those of skill in the art. In the Pichia system, commercially available vectors can be chosen from among those that are more suited for the production of cytosolic, non-glycosylated proteins and those that are more suited for the production of secreted, glycosylated proteins, or those directed to an intracellular organelle, so that appropriate protein expression can be optimized for the cargo moiety of choice that is a polypeptide.

The transport peptides of the present invention have the ability to cross the cell membrane of a cell (e.g., internalize into the cell). For example, in one embodiment of the invention, a transport peptide can translocate from the extracellular environment of a cell, penetrate the lipid bilayer of the cell membrane and cross the cell membrane into the intracellular environment of the cell. In another embodiment, the transport peptides of the present invention can selectively home to a target cell. In a further embodiment, the transport peptides can selectively home to and cross the cell membrane of a target cell. Selectively home is interpreted to mean a transport peptide that selectively binds to a target cell. A target cell is a specific cell type such as, for example, a cardiac cell, a skin cell (e.g., an endothelial cell), a skeletal muscle cell or a brain cell (e.g. a neuron) but may be any cell, including human and nonhuman cells.

The invention is useful for the delivery of cargo moieties across the cell membrane of a cell. The invention is also useful for the delivery of cargo moieties to a target cell (e.g., a specific cell type, such as a cardiac cell) and for the delivery of cargo moieties to a target cell and across the membrane of the target cell.

For example, in another embodiment of the invention, the transport peptides of the invention are linked to a cargo moiety and transport the cargo moiety across the cell membrane of a cell. For example, a (therapeutic) protein, such as caveolin or a transcription factor, linked to a transport peptide is carried from the extracellular environment of a cell and transported across the cell membrane and into the intracellular environment of the cell. In another embodiment of the invention, the transport peptide linked to a cargo moiety selectively homes the cargo moiety to a target cell (e.g., a cardiac cell). In a further embodiment of the present invention, the transport peptide linked to a cargo moiety selectively homes the cargo moiety to a target cell (e.g., a cardiac cell) and transports the cargo moiety from the extracellular environment of the target cell across the cell membrane and into the intracellular environment of the target cell.

The transport peptide linked to a cargo moiety, in an additional embodiment of the invention, is administered to an individual. In certain embodiments, the individual is a mammal such as a human. When administered to an individual, the transport peptide linked to a cargo moiety can be administered as a pharmaceutical composition containing, for example, the transport peptide linked to a cargo moiety and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters.

A pharmaceutically acceptable carrier can contain physiologically acceptable compounds that act, for example, to stabilize or to increase the absorption of the transport complex. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the route of administration of the composition.

One skilled in the art would know that a pharmaceutical composition containing a transport peptide linked to a cargo moiety can be administered to a subject by various routes including, for example, oral administration; intramuscular administration; intravenous administration; anal administration; vaginal administration; parenteral administration; nasal administration; intraperitoneal administration; subcutaneous administration and topical administration. The composition can be administered by injection or by intubation. The pharmaceutical composition also can be a transport peptide linked to a liposome or other polymer matrix, which can have incorporated therein, for example, a cargo moiety such as a drug that promotes or inhibits cell death (Gregoriadis, Liposome Technology, Vol. 1 (CRC Press, Boca Raton, Fla. 1984), which is incorporated herein by reference). Liposomes, for example, which consist of phospholipids or other lipids, are nontoxic, physiologically acceptable and metabolizable carriers that are relatively simple to make and administer.

The present invention is illustrated by the following examples, which are not intended to be limiting in any way.

### EXAMPLE 1

### Identification of Transport Peptides

Specific peptides were developed that can cross endothelial barriers and cross cell membrane barriers to allow delivery of genes and protein fusion constructs to targeted cells, in vivo and in vitro. These peptides were developed by a variety of methods and approaches. One such approach is the use of peptides phage display. Peptide phage display libraries were constructed consisting of T-7 bacteriophage that express random peptide sequences on their capsid. The libraries contain 108-109 unique peptide sequences, expressed as fusion constructs on a capsid protein. The libraries express random peptides 7-12 amino acids in length as fusions to the bacteriophage capsid.

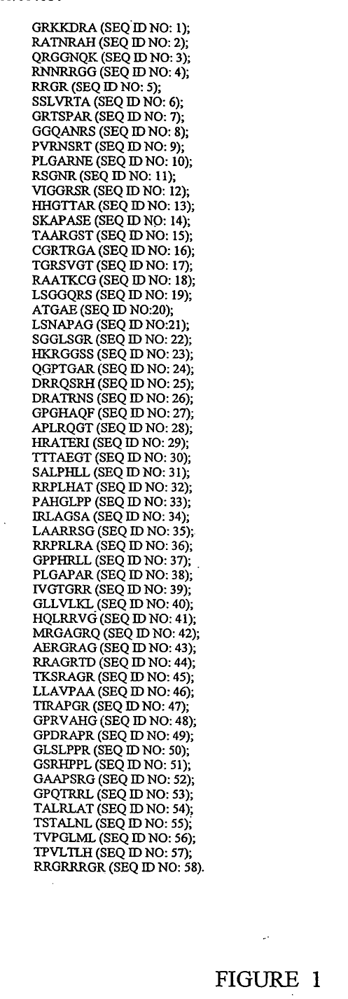

These libraries were used to perform functional biopanning experiments in which the internalization of the bacteriophage or the ability of the bacteriophage to cross endothelial barriers was used to select phage expressing unique peptides that directed these functional characteristics. Transport peptide sequences were defined by biopanning across endothelial cell monolayers grown on porous membrane filters. Those phage expressing peptide motifs that facilitated passage across the endothelial cell monolayer were rescued and amplified, and this process was repeated for enrichment. The sequences defined are the result of 6-7 rounds of biopanning in this manner. (See FIG. 1). Sequences capable of internalization were defined by a number of means, including phage uptake experiments in which the phage were incubated with cells, and the phage that internalized into the cells was rescued for recurrent rounds of enrichment.

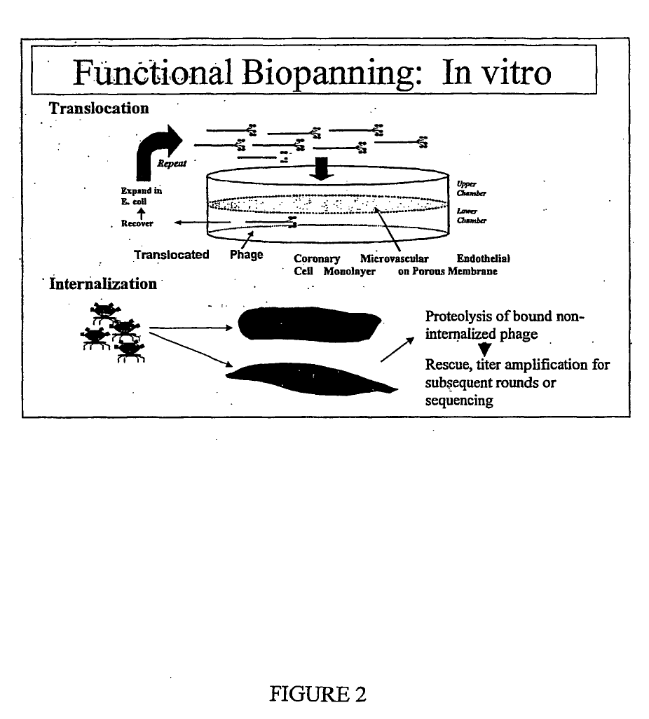

FIG. 2 shows diagrams of the in vitro approaches.

Another method used was to perfuse murine hearts ex-vivo on a Langendorf apparatus, administer the phage through the coronary circulation, and then rescue the phage that entered the myocardium. In vivo experiments were also done in which phage was injected into the general circulation of a mouse, any phage binding to the vasculature was removed by treatment with an enzyme solution, and then phage that had entered tissue parenchyma were rescued and enriched.

### EXAMPLE 2

### In Vitro Assessment of the Properties of Transport Peptides

To test the properties of the peptides defined, small scale synthesis of these peptides was ordered, labeled with rhodamine for fluorescent localization, from the Keck facility at Yale University. They were then used in in vitro and in vivo experiments to investigate the ability of these peptides to enter cells and tissues. The ability of these transport peptides to enter cells in culture was tested, and 100% transduction efficiency to these cultured cells was demonstrated.

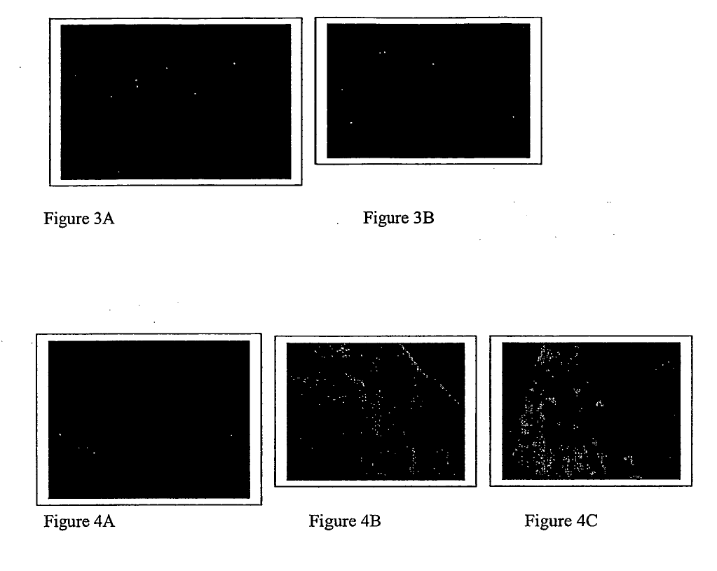

FIGS. 3A and 3B show pictures depicting highly efficient internalization of these peptides into cells in culture. One such transport peptide is RRGRRRGR. The pictures in FIGS. 3A and 3B demonstrate efficient uptake of an internalization peptide into endothelial cells (HUVEC cells)(FIG. 3A) and smooth muscle cells (FIG. 3B). Internalization of these peptides was demonstrated in rat aortic and adult human smooth muscle cells. Random peptides labeled in the same manner did not internalize.

### EXAMPLE 3

### In Vivo Assessment of the Properties of Transport Peptides

In experiments designed to test the ability of these peptides to cross the endothelium and enter cells in vivo, synthetic transport peptides labeled with the fluorescent marker rhodamine were infused into the coronary circulation of mice. To demonstrate the ability of these peptides to translocate into the heart after coronary infusion we infused the labeled peptides into the coronary circulation of mouse hearts. Peptides selected for internalization were capable of internalizing into the myocardium efficiently. The peptides exited the coronary circulation and entered the cardiac muscle with extreme efficiency that was not demonstrated with a control peptide of the same length (see FIGS. 4A-C).

The picture in FIG. 4A depicts virtually no uptake of a random (non-selected) peptide labeled with rhodamine. The pictures in FIGS. 4B and 4C depict efficient uptake of transport peptides (selected from the in vitro biopanning experiments) into the parenchyma of the heart after a single pass infusion through the coronary circulation. One such transport peptide is RRGRRRGR.

By incorporating these peptides into the capsid of gene-delivery viral vectors the efficiency of gene delivery by intracoronary infusion could be markedly enhanced. Additionally, electrostatic interaction of these peptides with the viral vectors may be enough to facilitate translocation of a cargo moiety, such as a viral vector, without actual covalent linkage.

### EXAMPLE 4

The protein, caveolin, interacts with endothelial nitric oxide synthase (eNOS). A peptide fragment of caveolin (cav) that contains only the caveolin-eNOS binding domain will block eNOS activity. Reduced eNOS activity leads to reduced vascular permeability.

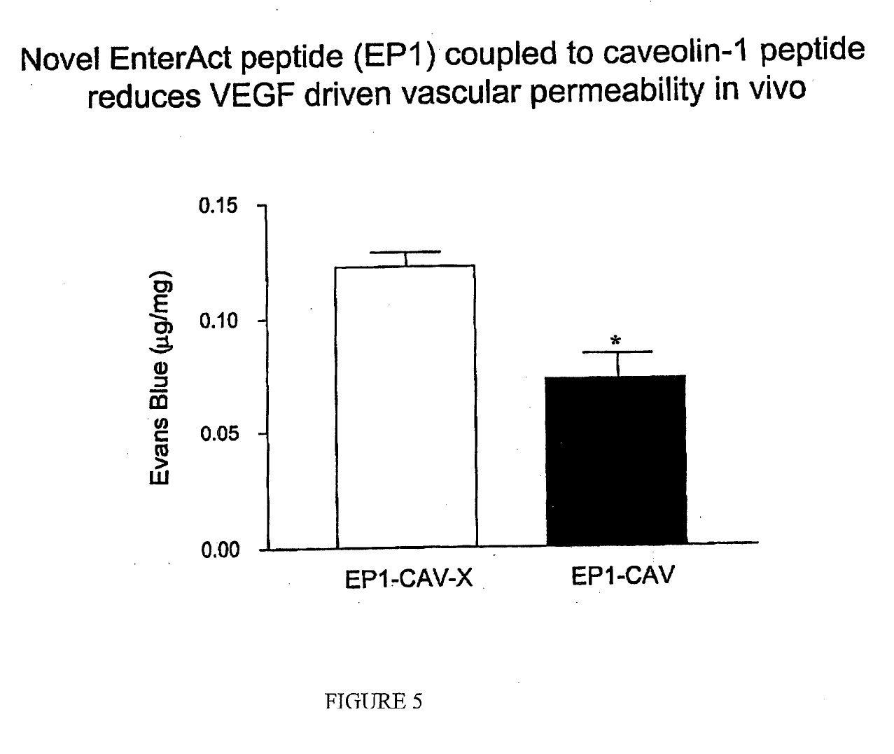

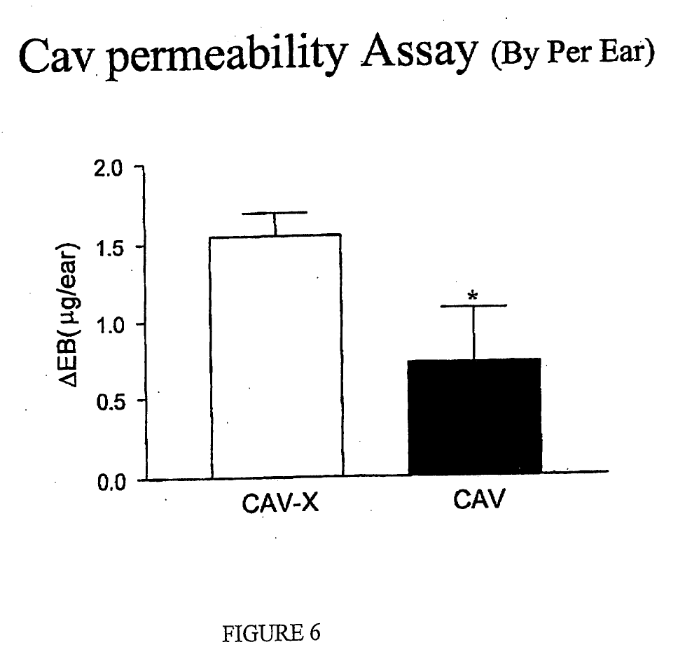

A transport peptide of the present invention, RGRRRGRR, was fused to a peptide fragment of caveolin (EP-cav) and to a mutant caveolin (EP-cav-x). Male Swiss mice (2530 grams) were pre-treated for 45 min with EP-cav or EP-Cav-X (2.5 mg/kg i.p. each). Animals were anesthetized with ketamine/xylazine, and a catheter was introduced into the left jugular vein for administration of Evans blue (30 mg/kg; Sigma). One minute following the administration of the dye, VEGF (300 ng) or saline was injected intradermally (30 ml total) into the right and left dorsal ear skin, respectively. After 30 minutes, animals were sacrificed and ears were removed, blotted dry, and weighed. Evans blue content of the ear was evaluated by extraction with 500 μl of formamide for 24 hours at 55° C. and measured spectrophotometrically at 610 nm. (See FIGS. 5 and 6).

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. Isolated nucleic acid encoding a peptide, wherein the amino acid sequence of the peptide is selected from the group consisting of:

| | | |
|---|---|---|
| (a) | GRKKDRA; | (SEQ ID NO: 1) |
| (b) | RATNRAH; | (SEQ ID NO: 2) |
| (c) | QRGGNQK; | (SEQ ID NO: 3) |
| (d) | RNNRRGG; | (SEQ ID NO: 4) |
| (e) | RRGR; | (SEQ ID NO: 5) |
| (f) | SSLVRTA; | (SEQ ID NO: 6) |
| (g) | GRTSPAR; | (SEQ ID NO: 7) |
| (h) | GGQANRS; | (SEQ ID NO: 8) |
| (i) | PVRNSRT; | (SEQ ID NO: 9) |
| (j) | PLGARNE; | (SEQ ID NO: 10) |
| (k) | RSGNR; | (SEQ ID NO: 11) |
| (l) | VIGGRSR; | (SEQ ID NO: 12) |
| (m) | HHGTTAR; | (SEQ ID NO: 13) |
| (n) | SKAPASE; | (SEQ ID NO: 14) |
| (o) | TAARGST; | (SEQ ID NO: 15) |
| (p) | CGRTRGA; | (SEQ ID NO: 16) |
| (q) | TGRSVGT; | (SEQ ID NO: 17) |
| (r) | RAATKCG; | (SEQ ID NO: 18) |
| (s) | LSGGQRS; | (SEQ ID NO: 19) |
| (t) | ATGAE; | (SEQ ID NO: 20) |
| (u) | LSNAPAG; | (SEQ ID NO: 21) |
| (v) | SGGLSGR; | (SEQ ID NO: 22) |
| (w) | HKRGGSS; | (SEQ ID NO: 23) |
| (x) | QGPTGAR; | (SEQ ID NO: 24) |
| (y) | DRRQSRH; | (SEQ ID NO: 25) |
| (z) | DRATRNS; | (SEQ ID NO: 26) |
| (aa) | GPGHAQF; | (SEQ ID NO: 27) |
| (bb) | APLRQGT; | (SEQ ID NO: 28) |
| (cc) | HRATERI; | (SEQ ID NO: 29) |
| (dd) | TTTAEGT; | (SEQ ID NO: 30) |
| (ee) | SALPHLL; | (SEQ ID NO: 31) |
| (ff) | RRPLHAT; | (SEQ ID NO: 32) |
| (gg) | PAHGLPP; | (SEQ ID NO: 33) |
| (hh) | IRLAGSA; | (SEQ ID NO: 34) |
| (ii) | LAARRSG; | (SEQ ID NO: 35) |
| (jj) | RRPRLRA; | (SEQ ID NO: 36) |
| (kk) | GPPHRLL; | (SEQ ID NO: 37) |
| (ll) | PLGAPAR; | (SEQ ID NO: 38) |
| (mm) | IVGTGRR; | (SEQ ID NO: 39) |
| (nn) | GLLVLKL; | (SEQ ID NO: 40) |
| (oo) | HQLRRVG; | (SEQ ID NO: 41) |
| (pp) | MRGAGRQ; | (SEQ ID NO: 42) |
| (qq) | AERGRAG; | (SEQ ID NO: 43) |
| (rr) | RRAGRTD; | (SEQ ID NO: 44) |
| (ss) | TKSRAGR; | (SEQ ID NO: 45) |
| (tt) | LLAVPAA; | (SEQ ID NO: 46) |
| (uu) | TIRAPGR; | (SEQ ID NO: 47) |
| (vv) | GPRVAHG; | (SEQ ID NO: 48) |
| (ww) | GPDRAPR; | (SEQ ID NO: 49) |
| (xx) | GLSLPPR; | (SEQ ID NO: 50) |
| (yy) | GSRHPPL; | (SEQ ID NO: 51) |
| (zz) | GAAPSRG; | (SEQ ID NO: 52) |
| (aaa) | GPQTRRL; | (SEQ ID NO: 53) |
| (bbb) | TALRLAT; | (SEQ ID NO: 54) |
| (ccc) | TSTALNL; | (SEQ ID NO: 55) |
| (ddd) | TVPGLML; | (SEQ ID NO: 56) |
| (eee) | TPVLTLH; | (SEQ ID NO: 57) |
| and | | |
| (fff) | RRGRRRGR. | (SEQ ID NO: 58) |

2. The isolated nucleic acid of claim 1, additionally comprising nucleic acid encoding a cargo moiety.

3. The isolated nucleic acid of claim 1, additionally comprising nucleic acid encoding at least two cargo moieties.

4. The isolated nucleic acid of claim 2, wherein the cargo moiety is selected from the group consisting of: (a) a peptide; (b) a protein; (c) a biologically active compound; (d) a drug; (e) a label; (f) an imaging agent; (g) a diagnostic agent; (h) a therapeutic agent; and (i) a prophylactic agent.

5. The isolated nucleic acid of claim 2, wherein the cargo moiety is caveolin or a fragment thereof.

6. Isolated peptide, wherein the amino acid sequence of the peptide is selected from the group consisting of:

| | | |
|---|---|---|
| (a) | GRKKDRA; | (SEQ ID NO: 1) |
| (b) | RATNRAH; | (SEQ ID NO: 2) |
| (c) | QRGGNQK; | (SEQ ID NO: 3) |
| (d) | RNNRRGG; | (SEQ ID NO: 4) |
| (e) | RRGR; | (SEQ ID NO: 5) |
| (f) | SSLVRTA; | (SEQ ID NO: 6) |
| (g) | GRTSPAR; | (SEQ ID NO: 7) |
| (h) | GGQANRS; | (SEQ ID NO: 8) |
| (i) | PVRNSRT; | (SEQ ID NO: 9) |
| (j) | PLGARNE; | (SEQ ID NO: 10) |
| (k) | RSGNR; | (SEQ ID NO: 11) |
| (l) | VIGGRSR; | (SEQ ID NO: 12) |
| (m) | HHGTTAR; | (SEQ ID NO: 13) |
| (n) | SKAPASE; | (SEQ ID NO: 14) |
| (o) | TAARGST; | (SEQ ID NO: 15) |
| (p) | CGRTRGA; | (SEQ ID NO: 16) |
| (q) | TGRSVGT; | (SEQ ID NO: 17) |
| (r) | RAATKCG; | (SEQ ID NO: 18) |
| (s) | LSGGQRS; | (SEQ ID NO: 19) |
| (t) | ATGAE; | (SEQ ID NO: 20) |
| (u) | LSNAPAG; | (SEQ ID NO: 21) |
| (v) | SGGLSGR; | (SEQ ID NO: 22) |
| (w) | HKRGGSS; | (SEQ ID NO: 23) |
| (x) | QGPTGAR; | (SEQ ID NO: 24) |
| (y) | DRRQSRH; | (SEQ ID NO: 25) |
| (z) | DRATRNS; | (SEQ ID NO: 26) |
| (aa) | GPGHAQF; | (SEQ ID NO: 27) |
| (bb) | APLRQGT; | (SEQ ID NO: 28) |
| (cc) | HRATERI; | (SEQ ID NO: 29) |
| (dd) | TTTAEGT; | (SEQ ID NO: 30) |
| (ee) | SALPHLL; | (SEQ ID NO: 31) |
| (ff) | RRPLHAT; | (SEQ ID NO: 32) |
| (gg) | PAHGLPP; | (SEQ ID NO: 33) |
| (hh) | IRLAGSA; | (SEQ ID NO: 34) |
| (ii) | LAARRSG; | (SEQ ID NO: 35) |
| (jj) | RRPRLRA; | (SEQ ID NO: 36) |
| (kk) | GPPHRLL; | (SEQ ID NO: 37) |
| (ll) | PLGAPAR; | (SEQ ID NO: 38) |
| (mm) | IVGTGRR; | (SEQ ID NO: 39) |
| (nn) | GLLVLKL; | (SEQ ID NO: 40) |
| (oo) | HQLRRVG; | (SEQ ID NO: 41) |
| (pp) | MRGAGRQ; | (SEQ ID NO: 42) |
| (qq) | AERGRAG; | (SEQ ID NO: 43) |
| (rr) | RRAGRTD; | (SEQ ID NO: 44) |
| (ss) | TKSRAGR; | (SEQ ID NO: 45) |
| (tt) | LLAVPAA; | (SEQ ID NO: 46) |
| (uu) | TIRAPGR; | (SEQ ID NO: 47) |
| (vv) | GPRVAHG; | (SEQ ID NO: 48) |
| (ww) | GPDRAPR; | (SEQ ID NO: 49) |
| (xx) | GLSLPPR; | (SEQ ID NO: 50) |
| (yy) | GSRHPPL; | (SEQ ID NO: 51) |
| (zz) | GAAPSRG; | (SEQ ID NO: 52) |
| (aaa) | GPQTRRL; | (SEQ ID NO: 53) |
| (bbb) | TALRLAT; | (SEQ ID NO: 54) |
| (ccc) | TSTALNL; | (SEQ ID NO: 55) |
| (ddd) | TVPGLML; | (SEQ ID NO: 56) |
| (eee) | TPVLTLH; | (SEQ ID NO: 57) |
| and | | |
| (fff) | RRGRRRGR. | (SEQ ID NO: 58) |

7. The isolated peptide of claim 6, wherein the peptide selectively homes to a target cell.

8. The isolated peptide of claim 6, wherein the peptide crosses the cell membrane of a cell.

9. The isolated peptide of claim 6, wherein the peptide: (a) selectively homes to a target cell and (b) crosses the cell membrane of the target cell.

10. A transport complex comprising a cargo moiety linked to a transport peptide, wherein the amino acid sequence of the transport peptide is selected from the group consisting of: SEQ ID NOS: 1-58.

11. The transport complex of claim 10, wherein the transport complex additionally comprises at least two cargo moieties.

12. The transport complex of claim 10, wherein the cargo moiety is selected from the group consisting of: (a) nucleic acid; (b) a peptide; (c) a protein; (d) an oligosaccharide; (e) a lipid; (f) a glycolipid; (g) a lipoprotein; (h) a biologically active compound; (i) a drug; (j) a label; (k) an imaging agent; (l) a diagnostic agent; (m) a therapeutic agent; (n) a prophylactic agent; and (O) a virus.

13. The transport complex of claim 10, wherein the cargo moiety is caveolin or a fragment thereof.

14. The transport complex of claim 10, wherein the transport complex homes to a target cell.

15. The transport complex of claim 10, wherein the transport complex crosses the cell membrane of a cell.

16. The transport complex of claim 10, wherein the transport complex: (a) selectively homes to a target cell; and (b) crosses the cell membrane of a target cell.

17. A pharmaceutical composition comprising the transport complex of claim 10 and a pharmaceutically acceptable carrier.

18. A vector comprising nucleic acid encoding a peptide, wherein the amino acid sequence of the peptide is selected from the group consisting of: SEQ ID NOS: 1-58.

19. The vector of claim 18 further comprising transcriptional activation elements sufficient for the expression of the nucleic acid encoding a peptide, wherein the amino acid sequence of the peptide is selected from the group consisting of: SEQ ID NOS: 1-58.

20. The vector of claim 19 additionally comprising nucleic acid encoding a cargo moiety in-frame with the nucleic acid encoding a peptide selected from the group consisting of: SEQ ID NOS: 1-58.

21. Isolated host cells comprising exogenous nucleic acid encoding a peptide, wherein the peptide has an amino acid sequence selected from the group consisting of: SEQ ID NOS: 1-58.

22. The host cells of claim 21, wherein the nucleic acid is a vector comprising: (a) nucleic acid encoding a peptide selected from the group consisting of: SEQ ID NOS: 1-58; and (b) nucleic acid encoding a cargo moiety in-frame with the nucleic acid encoding the peptide encoded in (a).

23. The host cells of claim 22, wherein the vector further comprises transcriptional activation elements sufficient for the expression of the nucleic acid of (a) and the nucleic acid of (b) in the host cells.

24. A method of producing a transport complex, wherein the amino acid sequence of the transport peptide is selected from the group consisting of: SEQ ID NOS: 1-58, comprising culturing host cells of claim 23 under conditions suitable for expression of the nucleic acid of (a) and the nucleic acid of (b), wherein a peptide selected from the group consisting of SEQ ID NOS: 1-58 linked to a cargo moiety is thereby produced.

25. A method of delivering a cargo moiety to a target cell comprising contacting the cell with a transport complex of claim 10, under conditions suitable for interaction of the transport complex with the target cell, wherein the cargo moiety linked to the transport peptide is delivered to the target cell.

26. A method of importing a cargo moiety into a cell comprising contacting the cell with a transport complex of claim 10, under conditions suitable for passage of the transport complex across the cell membrane and into the cell, wherein the cargo moiety linked to the transport peptide is imported into the cell.

27. The method of claim 25, further comprising importing a cargo moiety into the target cell, wherein the target cell is contacted with a transport complex of claim 10, under conditions suitable for passage of the transport complex across the target cell membrane and into the target cell, wherein the cargo moiety linked to the transport peptide is imported into the target cell.

28. The method of claim 25, wherein the cargo moiety is selected from the group consisting of: (a) nucleic acid; (b) a peptide; (c) a protein; (d) an oligosaccharide; (e) a lipid; (f) a glycolipid; (g) a lipoprotein; (h) a biologically active compound; (i) a drug; (j) a label; (k) an imaging agent; (l) a diagnostic agent; (m) a therapeutic agent; (n) a prophylactic agent; and (o) a virus.

29. The method of claim 26, wherein the cargo moiety is selected from the group consisting of: (a) nucleic acid; (b) a peptide; (c) a protein; (d) an oligosaccharide; (e) a lipid; (f) a glycolipid; (g) a lipoprotein; (h) a biologically active compound; (i) a drug; (j) a label; (k) an imaging agent; (l) a diagnostic agent; (m) a therapeutic agent; (n) a prophylactic agent; and (O) a virus.

30. The method of claim 27, wherein the cargo moiety is caveolin or a fragment thereof.

31. A method of delivering a cargo moiety into cells of an individual, comprising administering to the individual the pharmaceutical composition of claim 17.

32. The method of claim 31, wherein the pharmaceutical composition is administered by a route selected from the group consisting of: oral administration; intramuscular administration; intravenous administration; anal administration; vaginal administration; parenteral administration; nasal administration; intraperitoneal administration; subcutaneous administration and topical administration.

33. The method of claim 27, wherein the target cell is selected from the group consisting of: (a) a cardiac cell; (b) a skeletal muscle cell; (c) a skin cell; and (d) a brain cell.


SEQUENCE LISTING (59 sequences)

SEQ ID NO: 1 - protein, 7 aa, Artificial Sequence
  Transport peptide
GRKKDRA

SEQ ID NO: 2 - protein, 7 aa, Artificial Sequence
  Transport peptide
RATNRAH

SEQ ID NO: 3 - protein, 7 aa, Artificial Sequence
  Transport peptide
QRGGNQK

SEQ ID NO: 4 - protein, 7 aa, Artificial Sequence
  Transport peptide
RNNRRGG

SEQ ID NO: 5 - protein, 4 aa, Artificial Sequence
  Transport peptide
RRGR

SEQ ID NO: 6 - protein, 7 aa, Artificial Sequence
  Transport peptide
SSLVRTA

SEQ ID NO: 7 - protein, 7 aa, Artificial Sequence
  Transport peptide
GRTSPAR

SEQ ID NO: 8 - protein, 7 aa, Artificial Sequence
  Transport peptide
GGQANRS

SEQ ID NO: 9 - protein, 7 aa, Artificial Sequence
  Transport peptide
PVRNSRT

SEQ ID NO: 10 - protein, 7 aa, Artificial Sequence
  Transport peptide
PLGARNE

SEQ ID NO: 11 - protein, 5 aa, Artificial Sequence
  Transport peptide
RSGNR

SEQ ID NO: 12 - protein, 7 aa, Artificial Sequence
  Transport peptide
VIGGRSR

SEQ ID NO: 13 - protein, 7 aa, Artificial Sequence
  Transport peptide
HHGTTAR

SEQ ID NO: 14 - protein, 7 aa, Artificial Sequence
  Transport peptide
SKAPASE

SEQ ID NO: 15 - protein, 7 aa, Artificial Sequence
  Transport peptide
TAARGST

SEQ ID NO: 16 - protein, 7 aa, Artificial Sequence
  Transport peptide
CGRTRGA

SEQ ID NO: 17 - protein, 7 aa, Artificial Sequence
  Transport peptide
TGRSVGT

SEQ ID NO: 18 - protein, 7 aa, Artificial Sequence
  Transport peptide
RAATKCG

SEQ ID NO: 19 - protein, 7 aa, Artificial Sequence
  Transport peptide
LSGGQRS

SEQ ID NO: 20 - protein, 5 aa, Artificial Sequence
  Transport peptide
ATGAE

SEQ ID NO: 21 - protein, 7 aa, Artificial Sequence
  Transport peptide
LSNAPAG

SEQ ID NO: 22 - protein, 7 aa, Artificial Sequence
  Transport peptide
SGGLSGR

SEQ ID NO: 23 - protein, 7 aa, Artificial Sequence
  Transport peptide
HKRGGSS

SEQ ID NO: 24 - protein, 7 aa, Artificial Sequence
  Transport peptide
QGPTGAR

SEQ ID NO: 25 - protein, 7 aa, Artificial Sequence
  Transport peptide
DRRQSRH

SEQ ID NO: 26 - protein, 7 aa, Artificial Sequence
  Transport peptide
DRATRNS

SEQ ID NO: 27 - protein, 7 aa, Artificial Sequence
  Transport peptide
GPGHAQF

SEQ ID NO: 28 - protein, 7 aa, Artificial Sequence
  Transport peptide
APLRQGT

SEQ ID NO: 29 - protein, 7 aa, Artificial Sequence
  Transport peptide
HRATERI

SEQ ID NO: 30 - protein, 7 aa, Artificial Sequence
  Transport peptide
TTTAEGT

SEQ ID NO: 31 - protein, 7 aa, Artificial Sequence
  Transport peptide
SALPHLL

SEQ ID NO: 32 - protein, 7 aa, Artificial Sequence
  Transport peptide
RRPLHAT

SEQ ID NO: 33 - protein, 7 aa, Artificial Sequence
  Transport peptide
PAHGLPP

SEQ ID NO: 34 - protein, 7 aa, Artificial Sequence
  Transport peptide
IRLAGSA

SEQ ID NO: 35 - protein, 7 aa, Artificial Sequence
  Transport peptide
LAARRSG

SEQ ID NO: 36 - protein, 7 aa, Artificial Sequence
  Transport peptide
RRPRLRA

SEQ ID NO: 37 - protein, 7 aa, Artificial Sequence
  Transport peptide
GPPHRLL

SEQ ID NO: 38 - protein, 7 aa, Artificial Sequence
  Transport peptide
PLGAPAR

SEQ ID NO: 39 - protein, 7 aa, Artificial Sequence
  Transport peptide
IVGTGRR

SEQ ID NO: 40 - protein, 7 aa, Artificial Sequence
  Transport peptide
GLLVLKL

SEQ ID NO: 41 - protein, 7 aa, Artificial Sequence
  Transport peptide
HQLRRVG

SEQ ID NO: 42 - protein, 7 aa, Artificial Sequence
  Transport peptide
MRGAGRQ

SEQ ID NO: 43 - protein, 7 aa, Artificial Sequence
  Transport peptide
AERGRAG

SEQ ID NO: 44 - protein, 7 aa, Artificial SEquence
  Transport peptide
RRAGRTD

SEQ ID NO: 45 - protein, 7 aa, Artificial Sequence
  Transport peptide
TKSRAGR

SEQ ID NO: 46 - protein, 7 aa, Artificial Sequence
  Transport peptide
LLAVPAA

SEQ ID NO: 47 - protein, 7 aa, Artificial Sequence
  Transport peptide
TIRAPGR

SEQ ID NO: 48 - protein, 7 aa, Artificial Sequence
  Transport peptide
GPRVAHG

SEQ ID NO: 49 - protein, 7 aa, Artificial Sequence
  Transport peptide
GPDRAPR

SEQ ID NO: 50 - protein, 7 aa, Artificial Sequence
  Transport peptide
GLSLPPR

SEQ ID NO: 51 - protein, 7 aa, Artificial Sequence
  Transport peptide
GSRHPPL

SEQ ID NO: 52 - protein, 7 aa, Artificial Sequence
  Transport peptide
GAAPSRG

SEQ ID NO: 53 - protein, 7 aa, Artificial Sequence
  Transport peptide
GPQTRRL

SEQ ID NO: 54 - protein, 7 aa, Artificial Sequence
  Transport peptide
TALRLAT

SEQ ID NO: 55 - protein, 7 aa, Artificial SEquence
  Transport peptide
TSTALNL

SEQ ID NO: 56 - protein, 7 aa, Artificial Sequence
  Transport peptide
TVPGLML

SEQ ID NO: 57 - protein, 7 aa, Artificial Sequence
  Transport peptide
TPVLTLH

SEQ ID NO: 58 - protein, 8 aa, Artificial Sequence
  Transport peptide
RRGRRRGR

SEQ ID NO: 59 - protein, 9 aa, Artificial Sequence
  Sequence found routinely in receptors of the fibroblast growth factor family as well as related tyrosine kinase receptors
KKLLAVPAA